(19) United States
(12) Patent Application Publication
(10) Pub. No.: US 2004/0133104 A1
(43) Pub. Date: Jul. 8, 2004

(54) Detection of a coherent reflector in noisy ultrasound data

(76) Inventors: Claude Cohen-Bacrie, Paris (FR); Claire Levrier, Rueil-Malmaison (FR)

Correspondence Address: Philips Electronics North America Corporation, Corporate Patent Counsel, PO Box 3001, Briarcliff Manor, NY, 10510, US

(21) Appl. No.: 10/478,072

(22) PCT Filed: May 21, 2002
(86) PCT No.: PCT/IB02/01790

(30) Foreign Application Priority Data
May 22, 2001 (FR) 01/06747

(51) Int. Cl. A61B 8/00

(52) U.S. Cl. 600/437

(57) ABSTRACT

The invention relates to a method of analyzing a medium in order to label the zones of said medium as a function of the contents of said zones, which medium is exposed to ultrasound signals. The method includes a step for receiving echographic ultrasound signals by means of a set of elements, thus defining a set of received signals, a step for correlating at least one pair of signals which are received by two elements from among the set of elements and originate from at least one zone of said medium, a step for analyzing the maximum correlation value as a function of the distance between the two elements whereby the correlated signals are received, said analysis step enabling the detection of the presence of a coherent reflector in said zone of said medium, and a step for labeling the zone in conformity with the result of the analysis step.

The invention enables a binary image to be formed of a medium wherefrom noisy data originates, the binary values being attributed to the image as a function of the detection of a coherent reflector.

Application: Ultrasound apparatus, notably for use in the medical field.

The invention relates to a method of analyzing a medium in order to label zones of said medium as a function of the contents of said zone, said medium being exposed to ultrasound signals.

A method of this kind is known from the document WO 00/07035. The known method enables the labeling of an image of a medium exposed to by ultrasound signals while studying the intensities of the pixels of the image itself. Zones with strong noise, possibly corresponding to blood, are thus separated from zones with weaker noise which correspond to tissues.

The method disclosed in the cited document WO 00/07035 proposes the separation of the image zones with a different noise on the basis of data presented in the form of an image. It is a problem that the analysis of the medium is limited by the resolution in image pixels. Thus, it follows that the known method does not enable the localizing of coherent reflectors for example punctual reflectors within a noisy zone.

It is an object of the invention to provide a method which enables the analysis of a medium producing noisy ultrasound data so as to detect coherent reflectors therein.

The object of the invention is achieved by means of an analysis method of the kind set forth in the preamble which is characterized in that it includes:

a step for receiving echographic ultrasound signals by means of a set of elements, thus defining a set of received signals,

a step for correlating at least one pair of signals which are received by two elements from among the set of elements and which originate from at least one zone of said medium,

a step for analyzing the maximum correlation value as a function of the distance between the two elements whereby the correlated signals are received, said analysis step enabling the detection of the presence of a coherent reflector in said zone of said medium,

a step for labeling the zone in conformity with the result of the analysis step.

A coherent reflector results in a usually large maximum correlation value between neighboring echographic signals. Said maximum is then observed even for signals originating from receiving elements which are situated far apart. Conversely, the maximum correlation values for signals corresponding to noisy data (speckle zone) decrease linearly as a function of the distance between the receiving elements and ultimately disappears. This is the teaching of the Van Cittert Zernicke theorem. The invention proposes the analysis of said maximum correlation values for the detection of a coherent reflector in a medium which produces noisy data. The method in accordance with the invention thus enables a refined analysis of noisy zones (speckle zones) which are characteristic of conventionally acquired ultrasound images. The method in accordance with the invention thus enables better use of the data to be acquired by means of an echographic ultrasound method by utilizing a correlation of received signals instead of an analysis at a later stage of the image ultimately obtained.

According to an advantageous embodiment of the invention the analysis step consists in comparing the maximum correlation value with a given value. The given value is a criterion chosen in conformity with the detection precision to be achieved. The comparison with the given value is then carried out for distances between elements which are greater than a given distance, that is, in conformity with the manner to be described hereinafter.

According to a preferred embodiment of the invention, the labeling step leads to the formation of a binary image of the medium, the value 1 being assigned to the zones for which the presence of a coherent reflector is detected whereas the value 0 is assigned to the zones which present merely noisy data (speckle). This version is intended to enable the visualization of the information produced by the method in accordance with the invention, and can notably enable the use of this information, for example, for adapting the exposure of the medium to ultrasound so as to realize a different treatment of the zones in which a coherent reflector is detected.

According to the invention means for the detection of coherent reflectors in a medium producing noisy data can be included in any ultrasound apparatus. The invention can thus be advantageously used in the field of medical imaging and notably in the field of ultrasound imaging where the images obtained are customarily noisy.

One of the applications of the invention advantageously relates to the imaging by combined images (compound imaging) which consists in exposure of a medium to ultrasound in different directions and in combining the results so as to obtain a more complete and less noisy image. By enabling the localizing of coherent reflectors, otherwise not visible in a combined image, the invention also contributes to the refinement of the imaging results and can enable more exact and more correct diagnoses specifically in the case of medical imaging.

The invention will be described in detail hereinafter with reference to the accompanying diagrammatic drawings; therein:

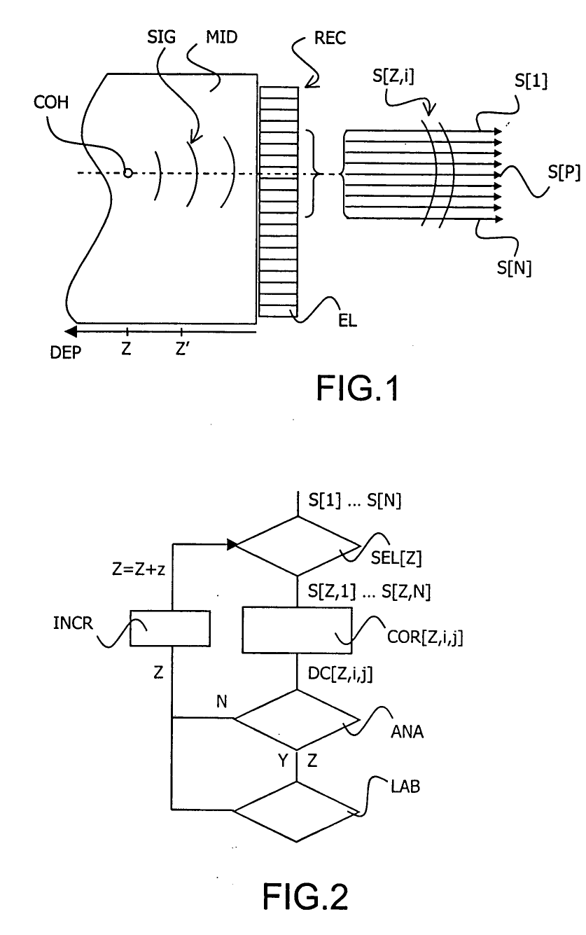

FIG. 1 shows a diagram illustrating the reception of signals from a medium in accordance with the invention,

FIG. 2 shows a functional diagram of an advantageous embodiment of the method for the analysis of a medium in accordance with the invention,

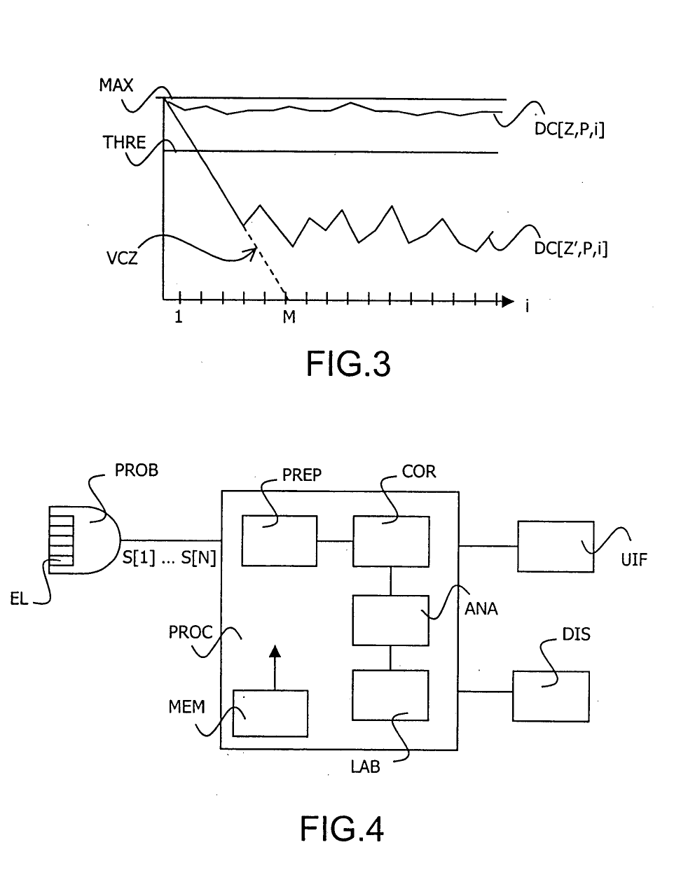

FIG. 3 shows a diagram illustrating a specific implementation of an analysis step in accordance with the invention, and

FIG. 4 is a diagrammatic representation of an apparatus in accordance with the invention.

The description given hereinafter is intended to enable a person skilled in the art to carry out and utilize the invention. Various alternatives to the preferred embodiment will be evident to a person skilled in the art and the general principles of the invention disclosed herein can be applied to other implementations. The present invention, therefore, is not considered to be limited to the embodiment described herein, but is intended to have the broadest scope in conformity with the principle and characteristics described hereinafter.

FIG. 1 shows a diagram illustrating the reception of echographic ultrasound signals in conformity with the invention. A medium MID, ultrasonically excited, emits echoes SIG. These echoes may originate from a medium which has a homogeneous structure and yields noisy signals (speckle zone) or may result from the reflection of an ultrasound wave from a coherent reflector COH which is situated in a zone at a mean depth DEP equal to Z. The problem to be solved consists in distinguishing the reflections from the medium which give rise to noisy signals (speckle) and those originating from the coherent reflector whose signals are buried in the noise (speckle). The signals are customarily received by a given number N of elements present in an array REC of elements. Generally speaking, the same array REC serves also for the emission. Customarily the excitation takes place for a finite number of elements (which may be equal to or different from N) of the array REC for which the excitation is carried out so as to focus the wave in a beam. In this case the medium is excited along a line. In FIG. 1 the line is centered in general on an element P which is situated at the center of the elements whereby the excitation is realized. The method in accordance with the invention aims to localize a coherent reflector on such a line. When a coherent reflector is detected on said line at said depth as determined by the method; the coherent reflector is completely localized. The reflection on such a reflector is translated into the propagation of a reflected signal SIG within the medium, said signal being received by the set of elements EL. This reflected signal arrives at each element of the array REC of elements at different instants. Each signal defines a set of received signals S[i], in this case being S[1] to S[N], that is, as many as there are elements active in the receiving mode. N elements EL are in effect activated for the reception in FIG. 1. The conventional analysis of this set of signals consists in applying an inverse focusing operation to the set of received signals so as to form an image subsequently. This operation comprises two steps: a shift in time of the signals received at different instants, necessary because of the different positions of the receiving elements with respect to the reflector, and a summing of the signals received by neighboring elements so as to reconstruct the echo signal. The invention proposes the use of the signals S[1] to S[N] as they arrive on the elements in order to detect the presence, if any, of a coherent reflector. The invention proposes to correlate the signals S[Z,i] with one another. The method in accordance with the invention advantageously utilizes the signals S[1] to S[N] after they have been shifted in time, that is, after the first step of the inverse operation described above. The signals S[i] are received during a given period of time and only a part of these signals S[Z,i] corresponds to the echo reflected by a zone situated at the mean depth Z. Thus, shifting the signals in time makes it easier to mark the signal parts S[Z,i].

FIG. 2 shows a functional diagram of an advantageous version of the analysis method for a medium in accordance with the invention. The signals S[1] to S[N] as received by the elements are processed during a first selection step SEL[Z] which selects the parts which correspond to a zone situated at a mean depth Z from among the signals. The selected signals S[Z,1] to S[Z,N] are subsequently correlated two by two in a step COR[Z,i,j]. Said step COR[Z,i,j] realizes the correlation of the signals originating from elements i and j. The maximum correlation values DC[Z,i,j] are obtained for each of the correlations COR[Z,i,j] carried out between the signals received by the elements i and j are obtained. Said maximum correlation values are subsequently analyzed in a step ANA so as to detect the presence of a coherent reflector on the line. Depending on the result of this analysis step, if a coherent reflector is detected (case Y), the zone wherefrom this information originates is given a label in a step LAB, after which the process analyzes another zone which corresponds to a mean depth Z=Z+z which is incremented in a step INCR. If no coherent reflector at all is detected (case N) in the analysis step ANA, the process directly increments the depth Z in the incrementation step INCR. This incrementation may be more or less large so as to probe the medium in a more or less refined manner. The analysis is subsequently carried out at the next depth and the process can thus be applied to all depths of a medium; an analysis of an entire slice of the medium is thus carried out.

FIG. 3 shows a diagram illustrating a specific implementation of an analysis step in accordance with the invention. The medium is exposed along a line which is centered on the element P which is situated centrally among the emission elements in the manner described with reference to FIG. 1. The correlation step can be carried out for all possible combinations of the various elements. A mean value of the maximum correlation values is then calculated for every feasible distance between two elements. This approach requires a large number of calculations.

In an advantageous embodiment, given with reference to FIG. 1, the correlation step takes place between an element i, where i is between 1 and N, and the element P which is situated at the center of said receiving elements; the same element, in this case the element P, is thus used for all correlations. This embodiment offers a saving as regards the given number of the calculations which would correspond to all possible combinations of the elements 1 to N.

In a further advantageous embodiment, a single correlation is performed between two signals received by elements situated at a known distance, said correlation being compared simply with a threshold. This version is very economical in respect of calculation work.

The analysis step can subsequently be performed on all values of the maximum correlation values DC obtained. FIG. 3 represents two specific cases of maximum correlation value curves DC as a function of the distance between the element P and the element i whose signal is correlated with that of the element P.

The curves DC[Z,P,i] and DC[Z′,P,i] correspond to two depths of the medium shown in FIG. 1. In this case they are presented in the context of the advantageous embodiment where an element P is used for each correlation, the element P thus being situated at the start of the curve. The depth Z corresponds to a coherent reflector COH and the depth Z′ to a medium resulting in a noisy signal (speckle). In conformity with the Van Cittert Zernicke theorem, the correlation of ultrasound signals received from a noisy zone (speckle zone) by neighboring elements is large for a distance zero (correlation with the same element) and decreases linearly until ideally it disappears for a distance between the two elements which correspond to the aperture used for the emission. In this case the curve thus disappears for a distance between two elements which is equal to the width existing between the elements activated in the emission mode. For example, as is shown in FIG. 3, if M elements are activated in the emission mode, the distance between two elements for which the maximum correlation value disappears is ideally M. The decrease between these two maximum correlation values is linear in conformity with the line VCZ shown in FIG. 3. In reality, depending on the level of the noise of the signals and the method of implementation of the correlation, the decrease of the maximum correlation values as a function of the distance of the elements approaches more or less exactly said ideal line VCZ (see curve DC[ZP,i]).

On the other hand, for a coherent reflector which is situated at the depth Z, the curve DC[Z,P,i] concerning the maximum correlation values as a function of the distance between the elements for which the signals received are correlated, remains close to the maximum MAX of the maximum correlation value obtained for the correlation of a received signal with itself. On the basis of these observations, a particularly advantageous implementation of the analysis step consists in the comparison of these curves with a threshold value THRE for a distance greater than the width of the aperture between the two elements for which the signals are correlated. When the curve exceeds the threshold value, the analysis step considers a reflector to be detected and, conversely, when the curve is below the threshold value, no coherent reflector at all is detected at the depth considered. Another particularly simple and advantageous embodiment consists in the calculation of a single correlation between two arbitrary receiving elements which, however, are situated a known distance apart, for example, M elements apart, where M is the number of elements activated in the emission mode, and in the comparison of the value MAX of the maximum correlation value obtained with the ideal value envisaged by the Van Cittert Zernicke theorem. If the maximum correlation value is close to the maximum that is possible for the maximum correlation values, a coherent reflector is detected. If the maximum correlation value is near the ideal curve VCZ, the zone is considered to be a noisy zone. In this case a threshold value with which the value of the correlation maximum obtained is compared can also be introduced; this threshold value is controlled as a function of the positions of the receiving elements whereby the correlated signals are received. For example, the threshold value will differ as the distance between the elements is such that the case analyzed is situated in the linear part VCZ or in the zero part of the curve of maximum correlation values. The detection can also take place on the basis of any calculation starting from the curve (calculation of the surface area of the triangle below the curve . . . ; this value of the surface area is significant and may also be compared with a threshold value). The depths Z for which a coherent reflector is detected are then given a label. In a preferred version this may lead to the formation of a binary image between the zones which corresponds to a medium which produces noisy signals and the zones in which a coherent reflector is detected. This binary image may simply be informative or may serve for carrying out modifications as regards the ultrasound exposure of the medium. Actually, the fact that it is known that a small reflector is present in a zone of the medium enables adaptation of the beam, for example, for increasing the visibility of the echo produced by this reflector by particularizing the focusing or the shape of the applied ultrasound wave.

FIG. 4 shows diagrammatically an apparatus in which a method in accordance with the invention is carried out. This apparatus includes a probe PROB which includes receiving elements EL and which is connected, by way of conventional means, to a conventional data processing apparatus PROC. Moreover, conventional data processing means DAT being provided, said data processing apparatus PROC advantageously includes a module for the preparation of signals PREP which shifts in time the signals S[1] to S[N] received by the elements EL of the probe PROB. Generally speaking, this step is performed in any ultrasound apparatus, after which the echo of the medium is reconstructed. It is generally followed by a step for summing the signals, which step is carried out by conventional data processing means DAT and is intended to reverse the focusing of the ultrasound exposure of the medium. In accordance with the invention the data processing means include selection means SEL whose function is as described with reference to FIG. 2. Said selection means enable the selection of parts of signals corresponding to zones of given mean depth of the medium. Finally, the apparatus PROC includes means COR for the correlation of signals, means ANA for the analysis of the maximum correlation values, and labeling means LAB. The apparatus PROC also includes storage means MEM, which storage means are used inter alia for the calculation operations. The apparatus PROC is connected to a display module DIS which enables the display, using conventional display functions, of images which can be constructed on the basis of information contained in the signals S[1] to S[N] by the invention, that is, in addition to the conventional images obtained by means of an ultrasound apparatus. It is actually possible that the process is linked to the display module so as to display a binary image constructed by means of conventional display means, said binary image being formed on the basis of labeled zones. The invention thus enables a binary image to be obtained from a medium producing noisy data, said binary values being attributed to the image as a function of the detection of a coherent reflector. A user interface UIF is advantageously connected to the apparatus PROC so as to control this apparatus and its parameters; for example, the modification of the threshold value which may be presented for modification by the user as well as the incrementation value for the depth which determines the precision of the detection as pursued by the invention.

The invention enables enhanced detection of coherent reflectors in a homogeneous medium for which noisy signals (speckle) are obtained; in such signals it is generally difficult to detect coherent reflectors by means of known means.

The modules presented above for carrying out the functions disclosed in the steps of the method in accordance with the invention may be integrated as an additional application in a conventional ultrasound apparatus or be implemented in an independent apparatus which is intended to be connected to a conventional ultrasound apparatus so as to carry out the functions in accordance with the invention. There are various ways of carrying out the functions presented in the steps of the methods in accordance with the invention, that is, by way of software and/or hardware means accessible to a person skilled in the art. Therefore, the Figures are shown merely diagrammatically. Thus, even though the Figures show different functions realized by different blocks, it is not excluded that a single software and/or hardware means enables several functions to be implemented. This does not exclude a combination of software and/or hardware means enabling the implementation of a function either. Even though the invention has been described with reference to the above embodiments, a person skilled in the art will immediately recognize that there are various alternatives to the embodiments presented and that these embodiments remain within the idea and the scope of the present invention. Numerous modifications can thus be realized by a person skilled in the art without departing from the teaching and the scope defined in the following claims.

1. A method of analyzing a medium in order to label zones of said medium as a function of the contents of said zones, said medium being excited by means of ultrasound signals, characterized in that the method includes:
a step for receiving echographic ultrasound signals by means of a set of elements, thus defining a set of received signals,
a step for correlating at least one pair of received signals which are received by two elements from among the set of elements and originate from at least one zone of said medium,
a step for analyzing the maximum correlation value as a function of the distance between the two elements whereby the correlated signals are received, said analysis step enabling the detection of the presence of a coherent reflector in said zone of said medium, and
a step for labeling the zone in conformity with the result of the analysis step.

2. An analysis method as claimed in claim 1, characterized in that the step for the analysis of the maximum correlation value consists in comparing the maximum correlation value with a given value.

3. A analysis method as claimed in one of the claims 1 or 2, characterized in that the labeling step leads to the formation of a binary image of the medium from which results a noisy signal, a first value being assigned to the zones in which a coherent reflector is detected whereas a second value is assigned to the other zones of the medium.

4. An apparatus which is intended for the analysis of a medium by labeling zones of said medium as a function of the contents of said zones, said medium being excited by means of ultrasound signals, which apparatus is characterized in that it includes:
means for receiving said ultrasound signals by means of a set of elements, thus defining a set of received signals,
means for correlating at least one pair of signals from among a set of received signals from at least one zone of said medium,
means for the analysis of the maximum correlation value as a function of the distance between the two elements whereby the correlated signals are received, said analysis step enabling the detection of the presence of a coherent reflector in said zone of said medium,
means for labeling the zone in conformity with the result of said analysis.

5. An apparatus as claimed in claim 4, which apparatus also includes means for adapting the ultrasound exposure of the medium as a function of the result of the analysis of the medium.

6. A computer program which is intended to be executed by a processor incorporated in an apparatus as claimed in one of the claims 4 and 5, characterized in that it contains a set of instructions for executing the steps of an analysis process as claimed in one of the claims 1 to 3.
